(19) United States
(12) Patent Application Publication
(10) Pub. No.: US 2026/0234114 A1
(43) Pub. Date: Aug. 13, 2026

(54) HAPTENS AND ANTIGENS FOR DETECTING XYLAZINE AND METHODS FOR SYNTHESIZING THE SAME

(71) Applicant: QINGDAO HENDERSON BIOLOGICAL TECHNOLOGY CO., LTD., Qingdao (CN)

(72) Inventors: Jianbin XIAO, Qingdao (CN); Chunhui FU, Qingdao (CN)

(73) Assignee: QINGDAO HENDERSON BIOLOGICAL TECHNOLOGY CO., LTD., Qingdao (CN)

(21) Appl. No.: 19/053,370

(22) Filed: Feb. 13, 2025

(51) Int. Cl. C07D 279/06; C07K 16/44; G01N 33/68; G01N 33/94

(52) U.S. Cl. CPC C07D 279/06; C07K 16/44; G01N 33/6854; G01N 33/9486

(57) ABSTRACT

Provided are haptens and antigens for detecting xylazine and methods for synthesizing the same, and the hapten is a compound of Formula (I) or a compound of Formula (II):

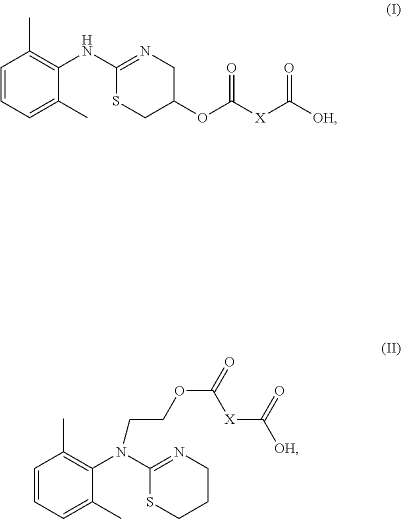

wherein X is an alkyl group having 0 to 10 carbon atoms.

### TECHNICAL FIELD

The present disclosure relates to the field of biotechnology, and in particular, to haptens and antigens for detecting xylazine and methods for synthesizing the same.

### BACKGROUND

The chemical name of xylazine is dimethylbenzeneamine thiazine, which is an α2-adrenergic receptor agonist with anticonvulsant, sedative, and potent analgesic properties, especially for excited and aggressive canines. When used alone, xylazine may be used for simple treatment or clinical examination of canines, such as wound treatment, bandaging, suturing, etc. When combined with other drugs for anesthesia, xylazine may enhance the anesthetic effect of the drugs and reduce toxic side effects of xylazine. Xylazine is soluble in 50 mg/mL methanol and also dissolves in dilute acid solutions.

Xylazine has significant effects on the cardiovascular system. Xylazine exhibits different effects on the cardiovascular system through different routes of administration. In some species, intravenous or intramuscular injection of xylazine may cause a transient arterial hypertension, followed by a relatively long period of hypotension and bradycardia.

In the 1960s, xylazine was approved by the U.S. Food and Drug Administration as a potent analgesic for veterinary use, and was not classified as a controlled substance. Due to its low cost and easy availability, xylazine has gradually become a substitute for some illicit stimulants. As of January 2023, new drug stimulants adulterated with xylazine have spread throughout the United States. Due to substantial harm to human tissues caused by xylazine and over 3,000 related deaths in the U.S., the White House Office of National Drug Control Policy classified xylazine-adulterated stimulants as an “emerging national threat” in April 2023. In addition, in May 2022, the first report of a death in Europe due to inhalation of xylazine-adulterated stimulants was recorded.

There are many techniques for detecting xylazine, such as high performance liquid chromatography, gas chromatography, gas chromatography-mass spectrometry, etc. Although these techniques have high detection sensitivity and may obtain accurate results, they have the drawbacks such as requiring expensive instrument and equipment, high requirements for testing materials, the need for purification and processing, etc.

Therefore, there is a need to provide haptens and antigens for detecting xylazine and methods for synthesizing the same, so as to rapidly and conveniently detecting xylazine in a subject.

### SUMMARY

One or more embodiments of the present disclosure provide a compound of Formula (I) or a compound of Formula (II) based on xylazine:

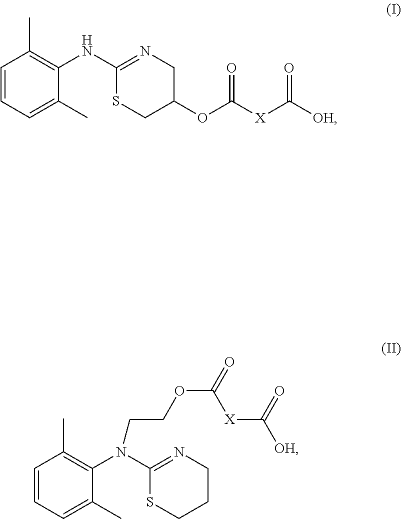

wherein X is an alkyl group having 0 to 10 carbon atoms.

One or more embodiments of the present disclosure provide a method of preparing a xylazine hapten of the compound of Formula (I), or a salt thereof, or a solvate thereof. The method includes: (a) adding o-tolyl isothiocyanate to anhydrous ethanol, adding 1-amino-3-chloro-2-propanol and ethyl acetate, and extracting an intermediate product, hydroxy xylazine; and (b) mixing the hydroxy xylazine with a cyclic anhydride compound, followed by adding toluene, to obtain the xylazine hapten of the compound of Formula (I), or the salt thereof, or the solvate thereof.

In some embodiments, in the operation (a), heating under reflux is performed after adding the 1-amino-3-chloro-2-propanol.

In some embodiments, in the operation (b), after adding the toluene, heating under reflux is performed followed by concentration.

In some embodiments, a molar ratio of the o-tolyl isothiocyanate to the 1-amino-3-chloro-2-propanol is 1:1. The cyclic anhydride compound is a glutaric anhydride, and a mass ratio of the hydroxy xylazine to the glutaric anhydride is 1:1.

One or more embodiments of the present disclosure provide a method for preparing a xylazine hapten of the compound of Formula (II), or a salt thereof, or a solvate thereof. The method includes: (A) adding xylazine and 2-iodoethanol to a N,N-Dimethylformamide (DMF) solution of K₂CO₃ to obtain an intermediate compound of Formula (III),

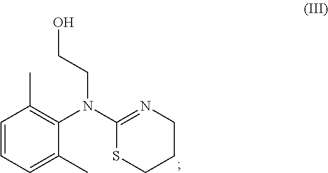 [SMILES: Cc1cccc(C)c1N(CCO)C1=NCCCS1]

and
(B) mixing the compound of Formula (III) with a cyclic anhydride compound, followed by adding toluene to obtain the xylazine hapten of the compound of Formula (II), or the salt thereof, or the solvate thereof.

Some embodiments of the present disclosure provide a xylazine antigen, obtained by conjugating the compound of Formula (I) or the compound of Formula (II) with a carrier protein.

In some embodiments, the carrier protein includes at least one selected from bovine serum albumin, ovalbumin, hemocyanin, thyroglobulin, and human serum albumin.

Some embodiments of the present disclosure provide a method for preparing the xylazine antigen provided in the present disclosure. The method includes: (i) adding the xylazine hapten to N,N-dimethylformamide, and adding 1-(3-dimethylaminopropyl)-3-ethylcarbodiimide hydrochloride and N-hydroxysuccinimide under stirring to obtain a hapten activation solution; (ii) dissolving a carrier protein in a phosphate-buffered saline (PBS) solution to obtain a carrier protein solution, and adding the hapten activation solution to the carrier protein solution under stirring to obtain the xylazine antigen; and (iii) removing unreacted xylazine hapten from the xylazine antigen by dialyzing the xylazine antigen with a PBS solution, centrifuging, and collecting a supernatant to obtain a xylazine-carrier protein antigen.

Some embodiments of the present disclosure provide a specific antibody prepared using a xylazine antigen, the specific antibody including at least one selected from a monoclonal antibody, a polyclonal antibody, a humanized antibody, a chimeric antibody, a recombinant antibody, a polyspecific antibody, a bispecific antibody, and an antibody fragment.

Some embodiments of the present disclosure provide a method for diagnosing whether xylazine is present in the body of a patient. The method includes: contacting the aforementioned specific antibody with a sample of the patient, and determining whether xylazine is present in the body of the patient by determining whether a xylazine-antibody complex is generated.

Some embodiments of the present disclosure provide a method for treating a patient containing xylazine, the method including: administering to the patient a therapeutically effective amount of the specific antibody.

Some embodiments of the present disclosure provide a method for preparing an anti-xylazine specific antibody. The method includes: stimulating a subject using the compound of Formula (I) or the compound of Formula (II), or the xylazine antigen, and collecting the anti-xylazine specific antibody from the subject.

Some embodiments of the present disclosure provide a method for detecting an anti-xylazine specific antibody, the method including: contacting the anti-xylazine specific antibody with the compound of Formula (I), or the compound of Formula (II), and detecting an amount of the anti-xylazine specific antibody based on a concentration of a generated hapten-antibody complex.

In some embodiments, the method for detecting the anti-xylazine specific antibody includes: contacting the anti-xylazine specific antibody with the xylazine antigen, and detecting the amount of the anti-xylazine specific antibody based on the concentration of the generated antigen-antibody complex.

Some embodiments of the present disclosure provide a composition for preparing a product for detecting xylazine, the composition including the compound of Formula (I) or the compound of Formula (II).

Some embodiments of the present disclosure provide a composition for preparing a product for detecting xylazine, the composition including the xylazine antigen described in the present disclosure.

Some embodiments of the present disclosure provide a composition for preparing a product for detecting xylazine, the composition including the specific antibody described in the present disclosure.

Some embodiments of the present disclosure provide a method for detecting xylazine. The method includes: contacting a test subject with the specific antibody described in the present disclosure, and determining whether the test subject contains xylazine by determining whether a xylazine-antibody complex is generated.

### BRIEF DESCRIPTION OF THE DRAWINGS

The present disclosure will be further illustrated by way of exemplary embodiments, which will be described in detail by means of drawings. These embodiments are not limited, and in these embodiments, the same numbering denotes the same structure, wherein:

FIG. 1 is a schematic diagram illustrating a process for preparing a xylazine hapten of a compound of Formula (I) according to Example 1 of the present disclosure;

FIG. 2 is a schematic diagram illustrating a process for preparing a xylazine hapten of a compound of Formula (II) according to Example 1 of the present disclosure;

FIG. 3 is a diagram of a mass spectrometry identification result of the xylazine hapten of the compound of Formula (I) prepared according to Example 1 of the present disclosure;

FIG. 4 is a diagram of a mass spectrometry identification result of the xylazine hapten of the compound of Formula (II) prepared according to Example 1 of the present disclosure;

FIG. 5 shows results of antiserum titer assay in immunized mice sera according to Example 4 of the present disclosure; and

FIG. 6 shows results of antibody specificity assay according to Example 4 of the present disclosure.

### DETAILED DESCRIPTION

In order to make the technical problem to be solved, technical solution and beneficial effects in the present disclosure clearer and more understandable, the technical solution of the present disclosure is further described in detail in the following in combination with embodiments. It should be understood that the specific embodiments described herein are only for explaining the present disclosure, and are not intended to limit the present disclosure.

All technical and scientific terms used in the present disclosure generally have the same meanings as are commonly understood by those skilled in the art, unless otherwise defined. As used in the present disclosure and claims, unless the context clearly suggests an exception, “a”, “an”, “the”, and/or “such” do not refer specifically to the singular, but also to the plural, unless the context clearly suggests an exception. For example, “one cell” includes two cells or a combination of more cells. Generally, the nomenclature used in the present disclosure and the laboratory procedures for cell culture, molecular genetics, organic chemistry, analytical chemistry, nucleic acid chemistry, and hybridization, as described hereinafter, are well known and commonly used in the art.

As used herein, the terms “about” and “approximately”, when referring to a number, generally refers to a number within a range of 1%, 5%, or 10% above or below (greater than or less than) the number, unless otherwise stated or clearly evident from the context (e.g., a number being less than 0% of a possible value or more than 100% of the possible value).

As used herein, the term “pharmaceutically acceptable salt of a compound” refers to salts within the scope of the present disclosure and include acid or base addition salts, which maintain the desired pharmacological activity and are biologically desirable (e.g., the salt does not exhibit excessive toxicity, allergenicity, or irritability, and is bioavailable). When a compound of the present disclosure has a basic group such as an amino group, the compound may form pharmaceutically acceptable salts with acids including: an inorganic acid (e.g., a hydrochloric acid, a boric acid, a nitric acid, a sulfuric acid, and a phosphoric acid), an organic acid (e.g., an alginate, a formic acid, an acetic acid, a benzoic acid, a gluconic acid, a fumaric acid, an oxalic acid, a tartaric acid, a lactic acid, a maleic acid, a citric acid, a succinic acid, a malic acid, a methanesulfonic acid, a benzenesulfonic acid, a naphthalenesulfonic acid, and a p-toluenesulfonic acid) or an acidic amino acid (e.g., aspartic acid and glutamic acid). When a compound of the present disclosure has an acidic group (e.g., a carboxylic acid group), the compound may form salts with: a metal, such as an alkali metal and an alkaline earth metal (e.g., Na⁺, Li⁺, K⁺, Ca²⁺, Mg²⁺, and Zn²⁺), an ammonia or organic amine (e.g., dicyclohexylamine, trimethylamine, triethylamine, pyridine, methylpyridine, ethanolamine, diethanolamine, and triethanolamine), or a basic amino acid (e.g., arginine, lysine, and ornithine). The pharmaceutically acceptable salts may be prepared in situ during isolation and purification of the compound, or prepared by reacting the purified compound in its free base or free acid form with a suitable acid or base, and isolating the resulting salt.

As used herein, the term “administration to a subject” includes introducing or delivering a compound to a subject through any route that performs the intended function of the compound, including but not limited to oral, intranasal, parenteral (intravenous, intramuscular, intraperitoneal, or subcutaneous), rectal, or topical administration. Administration includes both self-administration and administration by another person.

As used herein, the term “antibody” refers to an immunoglobulin or immunoglobulin-like molecule, which includes, but is not limited to, IgA, IgD, IgE, IgG, IgM, combinations thereof, similar molecules produced during the immune response in any vertebrate species (e.g., mammals, such as humans, goats, rabbits, and mice) and non-mammalian species.

As used herein, the term “antibody” (including an “intact immunoglobulin”) or “specific antibody” refers to an antibody that specifically binds to a target molecule (or a group of highly similar target molecules), thereby substantially excluding binding to other molecules. For example, a binding constant of an antibody or an antibody fragment to the target molecule in a biological sample is about 10³ M⁻¹ times, 10⁴ M⁻¹ times, or 10⁵ M⁻¹ times greater than a binding constant of the antibody or the antibody fragment to other molecules. The term “antibody” also includes genetically engineered forms of antibody, such as a chimeric antibody (e.g., a humanized mouse antibody), a heteroconjugate antibody (e.g., a bispecific antibody). Examples of antibodies include a monoclonal antibody, a polyclonal antibody, a humanized antibody, a chimeric antibody, a recombinant antibody, a polyspecific antibody, a bispecific antibody, and an antibody fragment. An antibody specifically binds to an antigen.

As used herein, the term “bispecific antibody” refers to an antibody that is capable of binding to two different antigens simultaneously. A bispecific antibody (BsAb) and a bispecific antibody fragment (BsFab) may include at least one arm that specifically binds an antigen (e.g., a tumor-associated antigen) and at least one other arm that specifically binds a targetable conjugate carrying a therapeutic or diagnostic agent.

As used herein, the term “antigen” refers to a molecule to which an antibody specifically binds. A target antigen may be a protein (e.g., an antigenic peptide), a carbohydrate, a nucleic acid, a lipid, a hapten, or other natural or synthetic compounds. The antigen may be administered to an animal subject to elicit an immune response in the animal subject.

As used herein, the term “effective amount” of a composition refers to an amount sufficient to achieve a desired prophylactic or therapeutic effect. For example, the “effective amount” may include an amount that results in a reduction in symptoms associated with a disease to be treated. The disease to be treated may include a disease or medical condition associated with a target polypeptide (e.g., breast cancer, colorectal cancer, brain cancer, etc.). The amount of composition administered to a subject depends on the severity, type and stage of the disease, as well as characteristics (e.g., general health status, age, gender, body weight and drug tolerance) of the subject. Those skilled in the art may determine the effective amount based on the characteristics of the subject and other factors. The compositions of the present disclosure may also be administered in combination with one or more other therapeutic compounds.

As used herein, the term “sample” refers to a clinical sample obtained from a subject or an isolated microorganism. In some embodiments, the sample is derived from an organism (i.e., a “biological sample”), such as a tissue, a body fluid, or a microorganism collected from a subject. The source of the sample includes, but is not limited to, mucus, sputum, bronchoalveolar lavage (BAL), bronchial wash (BW), whole blood, a body fluid, cerebrospinal fluid (CSF), urine, plasma, serum, or tissue.

As used herein, the term “specifically binding” refers to recognizing and binding to another molecule (e.g., an antigen) but substantially not recognizing and binding to other molecules. As used herein, the terms “specifically binding,” “specifically binding to” and/or “specific to” a particular molecule (e.g., an antigen or an epitope on an antigen) refer to binding to the particular molecule with a dissociation constant (Kd) of about 10⁻⁴M, 10⁻⁵M, 10⁻⁶M, 10⁻⁷M, 10⁻⁸M, 10⁻⁹M, 10⁻¹⁰M, 10⁻¹¹M, or 10⁻¹²M.

As used herein, the terms “subject”, “individual”, and “patient” are used interchangeably and refer to an individual organism, vertebrate, mammal, or human. In some embodiments, the individual, the patient, or the subject is a human.

As used herein, the term “containing xylazine in the body” refers to a state in which an individual has residual xylazine in the body as a result of the individual ingesting xylazine for various reasons, and “in the body” refers to in any cell or tissue in the body.

As used herein, the terms “treating” and “treatment” encompasses treating a disease or condition in a subject as described in the present disclosure and includes: (1) inhibiting the disease or condition, i.e., preventing the disease or condition from progressing; (2) mitigating the disease or condition, i.e., causing the disease or condition to subside; (3) slowing the progression of the disease or condition; and/or (4) suppressing, relieving, or mitigating one or more symptoms of the disease or condition.

As used herein, the term “hapten” refers to a partial or incomplete antigen, which is a substance that does not contain protein and not capable of stimulating antibody formation, but can react with antibodies. A hapten forms a conjugate with a high molecular weight immunogenic carrier, and the conjugate (i.e., immunogen) is subsequently administered into a human or animal subject.

As used herein, the term “immunogen” refers to a substance capable of triggering, producing, or generating an immune response in a host organism.

As used herein, the term “immunogenic carrier” refers to an immunogenic substance, usually a protein, that is capable of conjugating with a hapten at one or more sites to form an antigen, thereby stimulating the body to produce antibodies that specifically bind to the hapten. For example, immunogenic carriers includes, but is not limited to, a protein, a glycoprotein, a polyamino-polysaccharide complex, a particle, and a nucleic acid, which are considered exogenous substances capable of triggering an immune response in a host. The polyamino-polysaccharide complex may be prepared from a polysaccharide in any known conventional manner.

In some embodiments, a variety types of proteins may be used as immunogenic carriers, including, but not limited to, albumin, serum albumin, lipoproteins, or the like. For example, the proteins include bovine serum albumin, keyhole limpet hemocyanin, ovalbumin, bovine thyroglobulin, human serum albumin fraction V, rabbit albumin, pumpkin eed-globulin, diphtheria toxoid, tetanus toxoid, botulinum toxin, succinylated proteins, and synthetic polyamino acids such as polylysine. The immunogenic carrier may also include a polyamino-polysaccharide complex, which is a high molecular weight polymer formed through repeated condensation of monosaccharides. For example, polysaccharides may include starch, glycogen, cellulose, glycans such as gum arabic, agar, etc. Polysaccharide may also contain poly (amino acid) residues and/or lipid residues.

As used herein, the term “solvate” refers to an aggregate formed by a solute ion or molecule and one or more solvent molecules. The solvate may include, but is not limited to, an acetone solate, an ethanol solvate, a methanol solvate, a tert-butanol solvate (TBA solvate), a chloroform solvate, and other organic and inorganic solvate.

As used herein, the term “cyclic anhydride” or “cyclic anhydride compound” refers to a class of organic compounds characterized by a cyclic structure containing a carbonyl oxygen atom. The cyclic anhydride or cyclic anhydride compound is an anhydride form of a carboxylic acid. The molecular Formula of the cyclic anhydride is CₙH₂ₙO₃, wherein n represents the count of carbon atoms in the cyclic structure. The cyclic anhydride may be prepared by an anhydride reaction of a carboxylic acid.

In some embodiments, a linker group carrying an active functional group may be prepared by various manners. The linker group is used to connect a hapten to a carrier. The linker group may be formed using molecules with different functionalities, and upon activation of any end of the linker group, the linker group undergo sequential reactions with the hapten and the carrier. The type of functionality of the carrier to which the hapten is bound is determined by the hapten and the linker group that bind to the carrier.

Some embodiments of the present disclosure provide a compound of Formula (I) or a compound of Formula (II) based on xylazine:

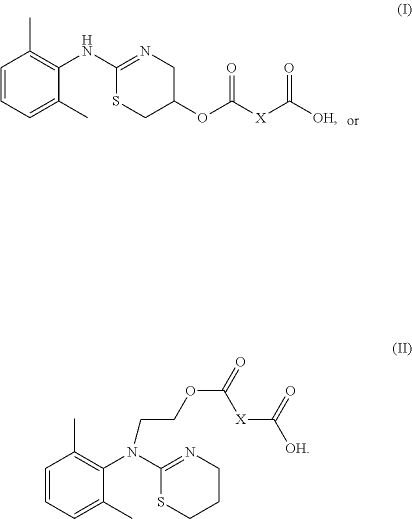

X is an alkyl group having 0 to 10 carbon atoms.

In some embodiments, X may be an alkyl group having 0, 1, 2, 3, 4, 5, 6, 7, 8, 9, or 10 carbon atoms. In some embodiments, X is an alkyl group having 3 carbon atoms.

In some embodiments, the compound of Formula (I) may be:

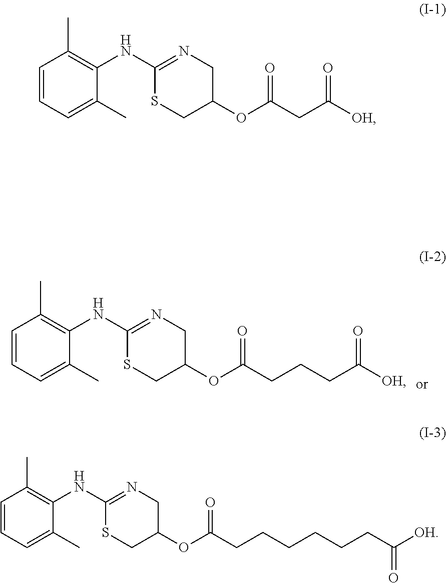 [SMILES: Cc1cccc(C)c1NC1=NCC(OC(=O)CC(=O)O)CS1 ; Cc1cccc(C)c1NC1=NCC(OC(=O)CCCC(=O)O)CS1 ; Cc1cccc(C)c1NC1=NCC(OC(=O)CCCCCCC(=O)O)CS1]

In some embodiments, the compound of Formula (II) may be:

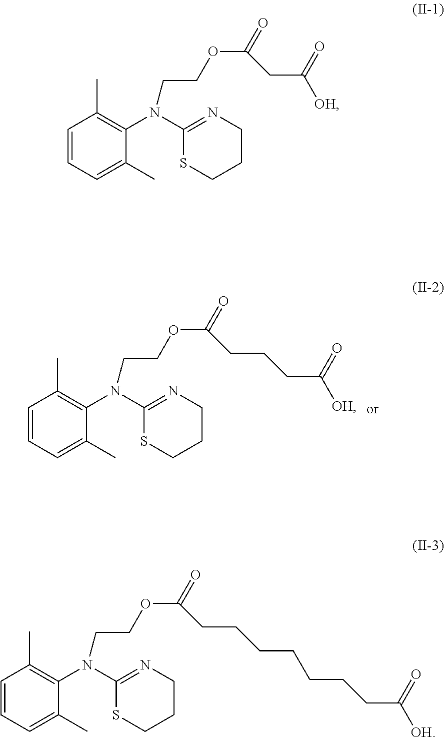 [SMILES: Cc1cccc(C)c1N(CCOC(=O)CC(=O)O)C1=NCCCS1 ; Cc1cccc(C)c1N(CCOC(=O)CCCC(=O)O)C1=NCCCS1 ; Cc1cccc(C)c1N(CCOC(=O)CCCCCCCC(=O)O)C1=NCCCS1]

Some embodiments of the present disclosure provide a method of preparing a xylazine hapten of the compound of Formula (I), or a salt thereof, or a solvate thereof. The method may include the following operations.

In operation (a), o-tolyl isothiocyanate is added to anhydrous ethanol, the mixture is dissolved, subsequently, 1-amino-3-chloro-2-propanol and ethyl acetate are added to obtain an intermediate product, hydroxy xylazine.

In some embodiments, a volume of the anhydrous ethanol is determined based on a standard that the anhydrous ethanol is capable of dissolving the o-tolyl isothiocyanate. In some embodiments, in operation (a), the dissolution temperature is room temperature.

In some embodiments, a molar ratio of o-tolyl isothiocyanate to 1-amino-3-chloro-2-propanol is 1:1.

In some embodiments, in operation (a), heating under reflux may be performed after adding 1-amino-3-chloro-2-propanol. In some embodiments, in operation (a), duration of heating under reflux is within a range of 1 hour to 6 hours.

In some embodiments, in operation (a), the duration of heating under reflux may be 1 hour, 2 hours, 3 hours, 4 hours, 5 hours, or 6 hours. In some embodiments, in operation (a), the duration of heating under reflux may be 6 hours.

In some embodiments, in operation (a), after heating under reflux, thin layer chromatography (TLC) is used to determine whether a reaction between o-tolyl isothiocyanate and 1-amino-3-chloro-2-propanol is complete.

In some embodiments, in operation (a), concentration may be performed after the heating under reflux. An intermediate crude product containing hydroxy xylazine is obtained after concentration.

After concentration, ethyl acetate is added to the intermediate crude product containing hydroxy xylazine. In some embodiments, an amount of the ethyl acetate is 5 mL.

In some embodiments, in operation (a), after adding the ethyl acetate, the mixture is stirred at room temperature for 30 minutes.

In some embodiments, in operation (a), the intermediate product, hydroxy xylazine, is obtained by removing the solvent through filtration.

In some embodiments, in operation (a), a molar ratio of the intermediate product, hydroxy xylazine, to the o-tolyl isothiocyanate is 3.6:6.13.

In some embodiments, a recovery rate of the intermediate product, hydroxy xylazine, obtained in operation (a) is within a range of 50%-70%. In some embodiments, the recovery rate of the intermediate product, hydroxy xylazine, obtained in operation (a) is 50%, 51%, 52%, 53%, 54%, 55%, 56%, 57%, 58%, 59%, 60%, 61%, 62%, 63%, 64%, 65%, 66%, 67%, 68%, 69%, or 70%. In some embodiments, the recovery rate of the intermediate product, hydroxy xylazine, obtained in operation (a) is 58.7%.

In operation (b), the xylazine hapten of the compound of Formula (I), or the salt thereof, or the solvate thereof may be obtained by mixing the hydroxy xylazine with a cyclic anhydride compound, followed by adding toluene.

In some embodiments, the cyclic anhydride compound may include succinic anhydride or glutaric anhydride. In some embodiments, the cyclic anhydride compound may be the glutaric anhydride, and a mass ratio of the hydroxy xylazine to the glutaric anhydride is 1:1. In some embodiments, a volume of the toluene added in operation (b) is 5 mL.

In some embodiments, in operation (b), heating under reflux may be performed after adding the toluene.

In some embodiments, in operation (b), duration of the heating under reflux is within a range of 1 hour −3 hours. In some embodiments, in operation (b), the duration of the heating under reflux may be 1 hour, 2 hours, or 3 hours. In some embodiments, in operation (b), the duration of the heating under reflux may be 3 hours.

In some embodiments, in operation (b), after the heating under reflux, TLC is used to determine whether a reaction between the hydroxy xylazine and the cyclic anhydride compound is complete.

In some embodiments, in operation (b), concentration may be performed after the heating under reflux. An intermediate crude product containing xylazine hapten is obtained after concentration.

In some embodiments, in operation (a), pure water may be added to the intermediate crude product containing the xylazine hapten after concentration. In some embodiments, in operation (b), a volume of the pure water may be 5 mL.

In some embodiments, in operation (b), stirring for 30 minutes at room temperature is performed after adding the pure water.

In some embodiments, in operation (b), a molar ratio of of the hydroxy xylazine to the xylazine hapten is 0.42:0.34.

In some embodiments, in operation (b), purified xylazine hapten may be obtained by removing the solvent through filtration.

In some embodiments, a recovery rate of the xylazine hapten obtained in operation (b) is within a range of 80%-90%. In some embodiments, the recovery rate of the xylazine hapten obtained in operation (b) may be 80%, 81%, 82%, 83%, 84%, 85%, 86%, 87%, 88%, 89%, or 90%. In some embodiments, the recovery rate of the xylazine hapten obtained in operation (b) may be 81.6%.

Merely by way of example, the method of preparing the xylazine hapten of the compound of Formula (I), or the salt thereof, or the solvate thereof in the present disclosure includes: 1) adding 6.13 mmol of o-tolyl isothiocyanate (1.00 g) to 30 mL of anhydrous ethanol, and stirring at room temperature until the o-tolyl isothiocyanate is dissolved; 2) adding 6.13 mmol of 1-amino-3-chloro-2-propanol (0.67 g), heating the mixture under reflux with magnetic stirring for 6 hours, with completion of the reaction confirmed by thin-layer chromatography; 3) obtaining an intermediate crude product containing hydroxy xylazine by concentrating the reaction solution under reduced pressure; 4) adding 5 mL of ethyl acetate to the intermediate crude product containing hydroxy xylazine, stirring at room temperature for 30 minutes, and removing the solvent by filtration to obtain an intermediate product, hydroxy xylazine; 5) adding 5 mL of toluene to 100 mg of the intermediate product, hydroxy xylazine (0.42 mmol) and 100 mg of glutaric anhydride (0.88 mmol), heating under reflux with magnetic stirring for 3 hours, with completion of the reaction confirmed by thin-layer chromatography; 6) obtaining an intermediate crude containing xylazine hapten by concentrating the reaction mixture under reduced pressure; (7) adding 5 mL of purified water to the intermediate crude containing xylazine hapten, stirring at room temperature for 30 minutes, and removing the solvent by filtration to obtain the purified xylazine hapten.

Some embodiments of the present disclosure provide a method of preparing a xylazine hapten of a compound of Formula (II), or a salt thereof, or a solvate thereof. The method may include the following operations.

In operation (A), an intermediate compound of Formula (III) may be obtained by adding xylazine and 2-iodoethanol to a N,N-Dimethylformamide (DMF) solution of K₂CO₃.

The compound of Formula (III) is:

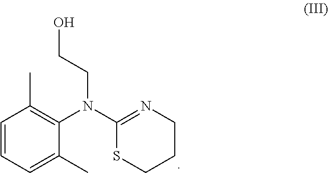 [SMILES: Cc1cccc(C)c1N(CCO)C1=NCCCS1]

In some embodiments, in operation (A), a mass of the xylazine is 100 mg, a mass of the 2-iodoethanol is 116.5 mg, and a volume of the DMF solution of K₂CO₃ is 3 mL.

In operation (B), the xylazine hapten of the compound of Formula (II), or the salt thereof, or the solvate thereof may be obtained by mixing the compound of Formula (III) with a cyclic anhydride compound, followed by adding toluene.

In some embodiments, in operation (B), heating under reflux may be performed after adding the toluene. Concentration is performed after heating under reflux. More details regarding the heating under reflux and concentration may be found in operation (a) and the related descriptions thereof.

Merely by way of example, the method for preparing the xylazine hapten of the compound of Formula (II), or the salt thereof, or the solvate thereof in the present disclosure includes the following operations: 1) adding xylazine (100 mg, 0.454 mmol) and 2-iodoethanol (116.5 mg, 0.681 mmol) to a DMF (3 mL) solution of K₂CO₃ (125 mg, 0.908 mmol), heating at 60° C. for 3 hours to complete the reaction, and performing post-treatment to obtain an intermediate product, the compound of Formula (III); 2) mixing the compound of Formula (III) with a cyclic anhydride compound, followed by adding toluene to obtain the xylazine hapten of the compound of Formula (II), or the salt thereof, or the solvate thereof.

Some embodiments of the present disclosure provide a xylazine antigen, which is obtained by conjugating the compound of Formula (I) or the compound of Formula (II) with a carrier protein.

In some embodiments, the carrier protein may include at least one selected from bovine serum albumin, ovalbumin, hemocyanin, thyroglobulin, and human serum albumin, or a combination thereof.

Some embodiments of the present disclosure provide a method of preparing a xylazine antigen. The method may include the following operations.

In operation (i), the xylazine hapten is added to N,N-dimethylformamide, and 1-(3-dimethylaminopropyl)-3-ethylcarbodiimide hydrochloride and N-hydroxysuccinimide are added under stirring to obtain a hapten activation solution.

In some embodiments, an amount of the N,N-dimethylformamide is determined based on a standard that N,N-dimethylformamide is capable of completely dissolving the xylazine hapten.

In some embodiments, in operation (i), the 1-(3-dimethylaminopropyl)-3-ethylcarbodiimide hydrochloride and the N-hydroxysuccinimide are added under stirring at room temperature.

In some embodiments, a molar ratio of the 1-(3-dimethylaminopropyl)-3-ethylcarbodiimide hydrochloride to the N-hydroxysuccinimide may be 1:1.

In some embodiments, in operation (i), after adding the 1-(3-dimethylaminopropyl)-3-ethylcarbodiimide hydrochloride and the N-hydroxysuccinimide, the mixture is stirred at room temperature for 4-6 hours.

In some embodiments, in operation (i), after adding the 1-(3-dimethylaminopropyl)-3-ethylcarbodiimide hydrochloride and the N-hydroxysuccinimide, duration of the stirring may be 4 hours, 5 hours, or 6 hours. In some embodiments, the duration of the stirring may be 5 hours.

In operation (ii), a carrier protein solution may be obtained by dissolving a carrier protein in a phosphate-buffered saline (PBS) solution, and the hapten activation solution may be added to the carrier protein solution under stirring to obtain the xylazine antigen.

In some embodiments, in operation (ii), the carrier protein may include at least one selected from bovine serum albumin, ovalbumin, hemocyanin, thyroglobulin, and human serum albumin, or a combination thereof.

In some embodiments, the carrier protein may include bovine serum albumin, keyhole limpethemocyanin, ovalbumin, bovine thyroglobulin, human serum albumin fraction V, rabbit albumin, pumpkin eed-globulin, diphtheria toxin, tetanus toxin, botulinum toxin, succinylated proteins, and synthetic poly(amino acids) such as polylysine.

In some embodiments, in operation (ii), the hapten activation solution is added to the carrier protein solution under stirring at room temperature.

In some embodiments, in operation (ii), after adding the hapten activation solution to the carrier protein solution, the mixture is stirred overnight.

In some embodiments, the overnight stirring is performed at room temperature.

In operation (iii), unreacted xylazine hapten is removed from the xylazine antigen by dialyzing the xylazine antigen with a PBS solution, followed by centrifugation, and a supernatant is collected to obtain a xylazine-carrier protein antigen.

In some embodiments, in operation (iii), the PBS has a pH value of 7.4 and a concentration of 0.01 mol/L.

In some embodiments, in operation (iii), duration of the centrifugation is within a range of 5 minutes-6 minutes, a rotational speed of the centrifugation is within a range of 6000 rpm-7000 rpm, and a temperature for the centrifugation is within a range of 2° C.-8° C.

In some embodiments, the xylazine-carrier protein antigen may include an immunogen and a coating antigen.

In some embodiments, when the carrier protein is bovine serum albumin, the xylazine-carrier protein antigen obtained by conjugating the bovine serum albumin to the xylazine hapten is a coating antigen or an immunogen. In some embodiments, when the carrier protein is hemocyanin, the xylazine-carrier protein antigen obtained by conjugating the hemocyanin to the xylazine hapten is a coating antigen or an immunogen.

Merely by way of example, the following embodiments describe the preparation of xylazine antigens using bovine serum albumin and keyhole limpet hemocyanin as carrier proteins, and the xylazine haptens of the compounds of Formula (I) and Formula (II).

In some embodiments, a xylazine antigen may be prepared by conjugating the compound of Formula (I) with bovine serum albumin (BSA) as the carrier protein through the following operations: 1) dissolving 0.029 mmol of the xylazine hapten of the compound of Formula (I) (10.0 mg) in 1 mL of DMF, adding 0.045 mmol of 1-(3-dimethylaminopropyl)-3-ethylcarbodiimide hydrochloride (8.5 mg) and 0.045 mmol of N-hydroxysuccinimide (5.1 mg) under stirring, and stirring at room temperature for 5 hours to obtain a hapten activation solution; 2) dissolving 38 mg of bovine serum albumin in 4 mL of a PBS solution (pH=7.4, 0.01 mol/L) to obtain a bovine serum albumin solution, adding the hapten activation solution to the bovine serum albumin solution under stirring, and stirring overnight at room temperature to obtain the xylazine antigen; 3) removing unreacted xylazine hapten from the xylazine antigen by sufficiently dialyzing the xylazine antigen with a PBS solution (pH=7.4, 0.01 mol/L) solution, centrifuging at 6000 rpm-7000 rpm for 5-6 minutes at 2° C.-8° C., and collecting a supernatant to obtain a xylazine-bovine serum albumin antigen (XYL-BSA-1) of the compound of Formula (I) as a coating antigen.

In some embodiments, a xylazine antigen may be prepared by conjugating the compound of Formula (I) with keyhole limpet hemocyanin (KLH) as the carrier protein through the following operations: 1) dissolving 0.014 mmol of the xylazine hapten of the compound of Formula (I) (5.0 mg) in 1 mL of N,N-dimethylformamide, adding 0.021 mmol of 1-(3-dimethylaminopropyl)-3-ethylcarbodiimide hydrochloride (5.0 mg) and 0.022 mmol of N-hydroxysuccinimide (2.5 mg) under stirring, and stirring at room temperature for 5 hours to obtain a hapten activation solution; 2) dissolving 20 mg of keyhole limpet hemocyanin in 4 mL of a PBS solution (pH=7.4, 0.01 mol/L) to obtain a keyhole limpet hemocyanin solution, adding the hapten activation solution to the keyhole limpet hemocyanin solution under stirring, and stirring overnight at room temperature to obtain the xylazine antigen; 3) removing unreacted xylazine hapten from the xylazine hapten by sufficiently dialyzing the xylazine hapten with a PBS solution (pH=7.4, 0.01 mol/L), centrifuging at 6000 rpm-7000 rpm for 5-6 min at 2° C.-8° C., and collecting a supernatant to obtain a xylazine-keyhole limpet hemocyanin antigen (XYL-KLH-1) of the compound of the Formula (I) as an immunogen.

In some embodiments, a xylazine antigen may be prepared by conjugating the compound of Formula (II) with bovine serum albumin as the carrier protein through the following operations: 1) dissolving 0.026 mmol of the xylazine hapten of the compound of Formula (II) (10.0 mg) in 1 mL of DMF, adding 0.045 mmol of 1-(3-dimethylaminopropyl)-3-ethylcarbodiimide hydrochloride (8.5 mg) and 0.045 mmol of N-hydroxysuccinimide (5.1 mg) under stirring, and stirring at room temperature for 5 hours to obtain a hapten activation solution; 2) dissolving 35 mg of bovine serum albumin in 4 mL of PBS (pH=7.4, 0.01 mol/L) to obtain a bovine serum albumin solution, adding the hapten activation solution to the bovine serum albumin solution under stirring, and stirring overnight at room temperature to obtain the xylazine antigen; 3) removing unreacted xylazine hapten from the xylazine antigen by sufficiently dialyzing the xylazine antigen with a PBS solution (pH=7.4, 0.01 mol/L), centrifuging at 6000-7000 rpm for 5-6 min at 2-8° C., and collecting a supernatant to obtain a xylazine-bovine serum albumin antigen (XYL-BSA-2) of the compound of Formula (II) as a coating antigen.

In some embodiments, a xylazine antigen may be prepared by conjugating the compound of Formula (II) with keyhole limpet hemocyanin as the carrier protein through the following operations: 1) dissolving 0.013 mmol of the xylazine hapten of the compound of Formula (II) (5.0 mg) in 1 mL of DMF, adding 0.021 mmol of 1-(3-dimethylaminopropyl)-3-ethylcarbodiimide hydrochloride (4.0 mg) and 0.022 mmol of N-hydroxysuccinimide (2.5 mg) under stirring, and stirring at room temperature for 5 hours to obtain a hapten activation solution; 2) dissolving 20 mg of keyhole limpet hemocyanin in 4 mL of a PBS solution (pH=7.4, 0.01 mol/L) to obtain a keyhole limpet hemocyanin solution, adding the hapten activation solution to the keyhole limpet hemocyanin solution under stirring, and stirring overnight at room temperature to obtain the xylazine antigen; 3) removing unreacted xylazine hapten from the xylazine antigen by sufficiently dialyzing the xylazine antigen with a PBS solution (pH=7.4, 0.01 mol/L), centrifuging at 6000-7000 rpm for 5-6 minutes at 2-8° C., and collecting a supernatant to obtain a xylazine-keyhole limpet hemocyanin antigen (XYL-KLH-2) of the compound of Formula (II) as an immunogen.

Some embodiments of the present disclosure provide a specific antibody prepared using a xylazine antigen, and the specific antibody may include at least one selected from a monoclonal antibody, a polyclonal antibody, a humanized antibody, a chimeric antibody, a recombinant antibody, a polyspecific antibody, a bispecific antibody, and an antibody fragment.

Some embodiments of the present disclosure provide a method for diagnosing whether xylazine is present in the body of a patient. The method includes contacting the specific antibody with a sample of the patient, and determining whether xylazine is present in the body of the patient by determining whether a xylazine-antibody complex is generated.

In some embodiments, the sample of the patient may include mucus, sputum, bronchoalveolar lavage (BAL), a bronchial wash liquid (BW), whole blood, a body fluid, a cerebrospinal fluid (CSF), urine, plasma, serum, tissue, etc.

Some embodiments of the present disclosure provide a method for treating a patient containing xylazine. The method includes administering to the patient a therapeutically effective amount of the specific antibody as described above.

In some embodiments, administration routes for the treatment may include oral, intranasal, parenteral (intravenous, intramuscular, intraperitoneal, or subcutaneous), rectal, or topical administration.

Some embodiments of the present disclosure provide a method for preparing an anti-xylazine specific antibody. The method includes stimulating a subject using the compound of Formula (I) or the compound of Formula (II), and collecting the anti-xylazine specific antibody from the subject.

In some embodiments, the subject may include a laboratory animal, such as a mouse. In some embodiments, the subject may also include a living tissue, a cell, or the like.

In some embodiments, the method may further include stimulating the subject using the xylazine antigen described above, and collecting the anti-xylazine specific antibody from the subject.

Some embodiments of the present disclosure provide a method for detecting an anti-xylazine specific antibody. The method includes: contacting the anti-xylazine specific antibody with the compound of Formula (I) or the compound of Formula (II), and detecting an amount of the anti-xylazine specific antibody based on a concentration of a generated hapten-antibody complex. In some embodiments, the method may include contacting the anti-xylazine specific antibody with the xylazine antigen described above, and detecting the amount of the anti-xylazine specific antibody based on the concentration of the generated antigen-antibody complex.

Some embodiments of the present disclosure provide a composition for preparing a product for detecting xylazine. The composition includes the compound of Formula (I) or the compound of Formula (II), the aforementioned xylazine antigen, and/or the aforementioned specific antibody.

Some embodiments of the present disclosure provide a method for detecting xylazine, the method including contacting a test subject with the specific antibody described above, and determining whether the test subject contains the xylazine by determining whether a xylazine-antibody complex is generated. In some embodiments, the test subject may be an organism. In some embodiments, the test subject may include a vertebrate, a mammal, or a human. In some embodiments, the test subject may include a living cell, a tissue, or the like.

The method of preparing the xylazine hapten and xylazine antigen provided in the embodiments of the present disclosure enable the rapid and efficient preparation of high-purity hapten or antigen. Additionally, the specific antibody prepared using the xylazine antigen for detecting xylazine offers advantages such as high titer, high sensitivity, and strong specificity. This allows for the rapid and convenient detection of xylazine, demonstrating significant potential for development and substantial market value in the field of detecting xylazine-based veterinary drugs.

In some embodiments of the present disclosure, whether a sample contains xylazine can be rapidly detected by immunochromatography.

The present disclosure is described in detail below in connection with specific examples. The following examples are described for those skilled in the art to further understand the present disclosure, but do not limit the scope of the present disclosure.

Quantitative assays in the following examples were conducted in triplicate, with the results averaged. Unless otherwise specified, the techniques used in the examples are conventional techniques well known to those skilled in the art, and all raw materials used were commercially available.

### EXAMPLES

### Example 1: Preparation and Characterization of a Xylazine Hapten

Example 1 provides a xylazine hapten having a structure of Formula (I) or Formula (II):

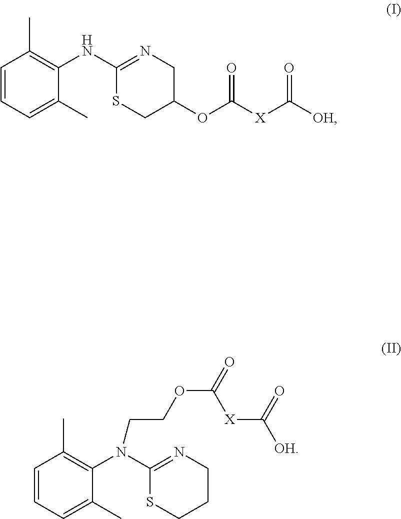

X is an alkyl group having 0 to 10 carbon atoms.

### 1. Preparation of the Xylazine Hapten of the Compound of Formula (I)

1) 6.13 mmol of o-tolyl isothiocyanate (1.00 g) was added to a 100 mL round-bottomed flask, and then 30 mL of anhydrous ethanol was added and stirred at room temperature until the o-tolyl isothiocyanate was dissolved.
(2) 6.13 mmol of 1-amino-3-chloro-2-propanol (0.67 g) was added and heated to reflux under magnetic stirring, the reflux was maintained for 6 hours, and whether the reaction between the o-tolyl isothiocyanate and the 1-amino-3-chloro-2-propanol was complete was determined by thin-layer chromatography.
3) An intermediate crude product containing hydroxy xylazine was obtained by concentrating the reaction mixture under reduced pressure.
4) 5 mL of ethyl acetate was added to the intermediate crude product containing hydroxy xylazine, the mixture was stirred at room temperature for 30 minutes, and a solvent was removed by filtering to obtain a purified intermediate product, hydroxy xylazine.
5) A mass of the intermediate product, hydroxy xylazine, was measured to be 0.85 g with a recovery rate of 58.7%.
(6) After mixing 100 mg of the intermediate product, hydroxy xylazine (0.42 mmol), with 100 mg of glutaric anhydride (0.88 mmol), 5 mL of toluene was added, the mixture was heated under reflux with magnetic stirring for 3 hours, and whether the reaction between the hydroxy xylazine and the glutaric anhydride was complete was determined by thin-layer chromatography.
7) An intermediate crude product containing xylazine hapten was obtained by concentrating under reduced pressure.
8) 5 mL of pure water was added to the intermediate crude product containing xylazine hapten, stirred at room temperature for 30 minutes, and the solvent was filtered to obtain a purified xylazine hapten.
9) A mass of the purified xylazine hapten was measured to be 120 mg with a recovery rate of 81.6%.

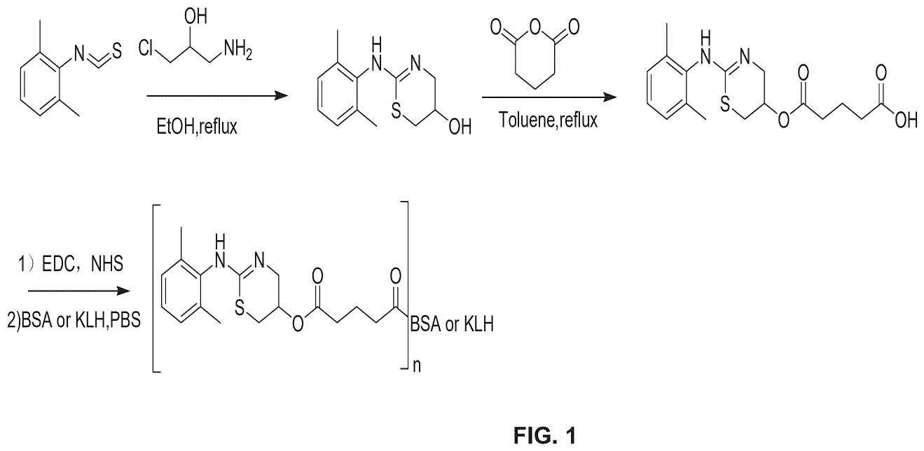

The process for preparing the xylazine hapten of the compound of Formula (I) is shown in FIG. 1.

### 2. Preparation of the Xylazine Hapten of the Compound of Formula (II)

1) Xylazine (100 mg, 0.454 mmol) and 2-iodoethanol (116.5 mg, 0.681 mmol) were added to a DMF (3 mL) solution of K₂CO₃ (125 mg, 0.908 mmol), the mixture was heated at 60° C. for 3 hours to obtain a reaction solution, which was added into 20 mL of iced water. The resulting mixture was extracted three times with 10 mL of ethyl acetate, and the organic layers were combined. The combined organic layer was washed once with 20 mL of saturated brine, dried over anhydrous sodium sulfate, and concentrated under reduced pressure to obtain an intermediate compound of Formula (III).
(2) After mixing the intermediate compound of Formula (III) (30 mg, 0.113 mmol) with glutaric anhydride (15.5 mg, 0.136 mmol), 5 mL of toluene was added.

The mixture was heated under reflux for 3 hours. After the toluene was removed by concentration under reduced pressure, 5 mL of purified water was added. The resulting mixture was extracted three times with 5 mL of ethyl acetate, and the organic layers were combined. The combined organic layer was washed once with 10 mL of saturated brine, dried over anhydrous sodium sulfate, and concentrated under reduced pressure to obtain a crude hapten product (41 mg).

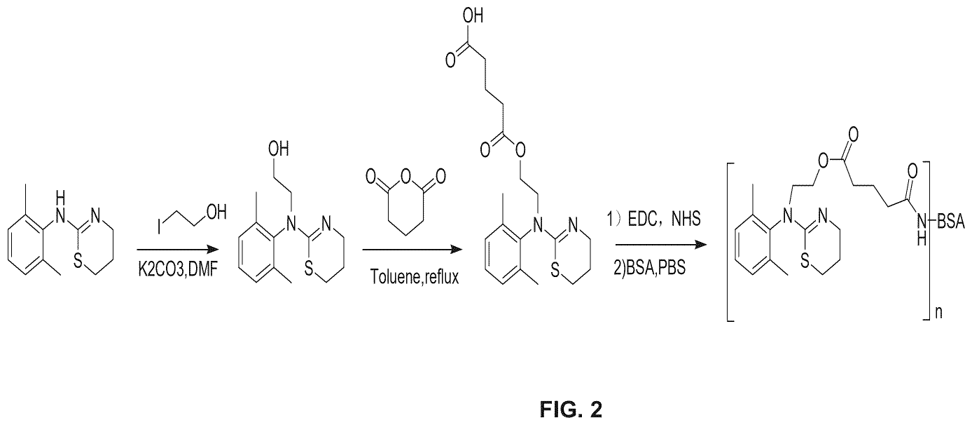

The process for preparing the xylazine hapten of the compound of Formula (II) is shown in FIG. 2.

### 3. Characterization of the Xylazine Hapten

The xylazine hapten of the compound of Formula (I) and the xylazine hapten of the compound of Formula (II) were characterized by mass spectrometry, respectively.

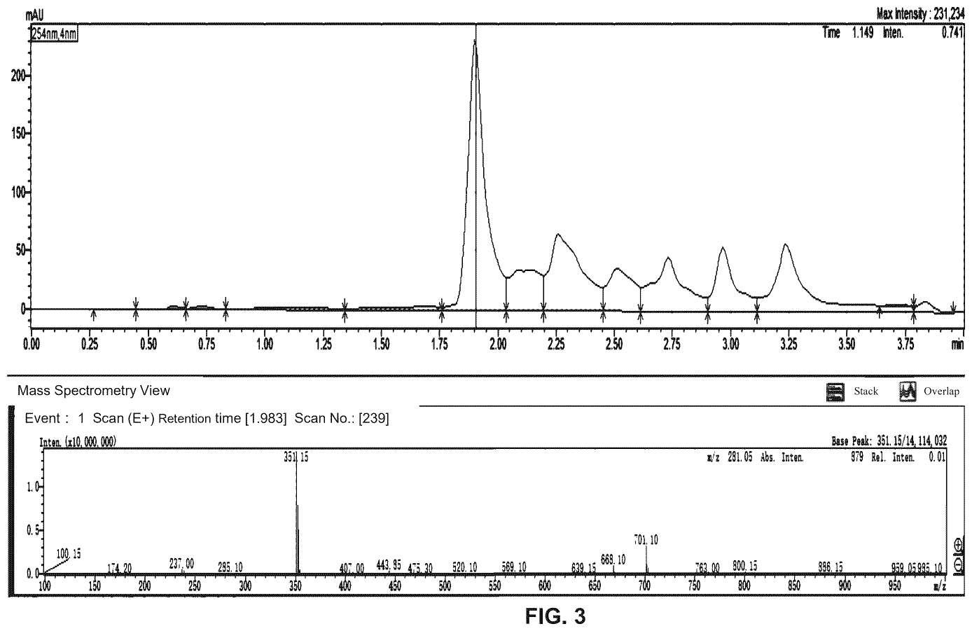

Mass spectrometry results for the xylazine hapten of the compound of Formula (I) is shown in FIG. 3. The detected mass is 351 (M+H)+, which is consistent with the theoretical mass of 350 for the target product.

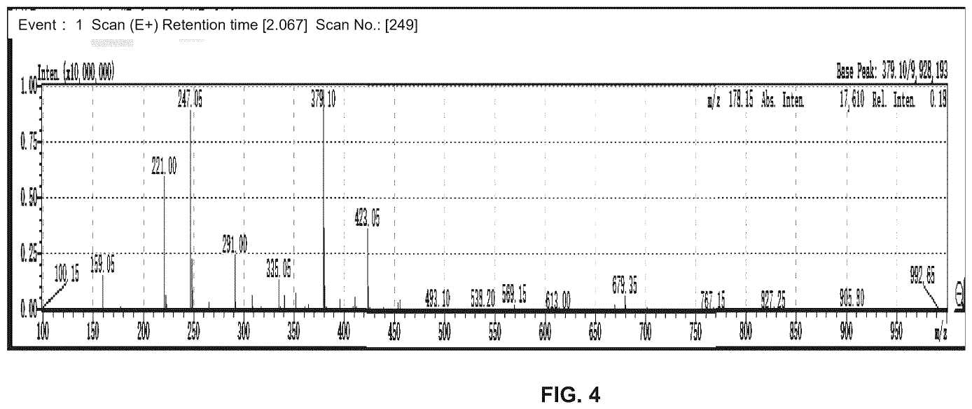

Mass spectrometry results for the xylazine hapten of the compound of Formula (II) is shown in FIG. 4. The detected mass is 379 (M+H)+, which is consistent with the theoretical mass of 378 for the target product.

### Example 2: Preparation and Characterization of an Artificial Xylazine Antigen

In Example 2, the artificial xylazine antigen is obtained by conjugating the xylazine hapten of the compound of Formula (I) or the xylazine hapten of the compound of Formula (II) with a carrier protein. The artificial xylazine antigen includes an immunogen and a coating antigen. The difference between a method of preparing the immunogen and a method of preparing the coating antigen is the type of the carrier protein used. The carrier protein used for the immunogen is mainly keyhole limpet hemocyanin, and the carrier protein used for the coating antigen is mainly bovine serum albumin.

### 1. Preparation of a Xylazine Coating Antigen of Formula (I)

(1) 0.029 mmol of the xylazine hapten (10.0 mg) of Formula (I) was dissolved in 1 mL of N,N-dimethylformamide, followed by stirring and adding 0.045 mmol of 1-(3-dimethylaminopropyl)-3-ethylcarbodiimide hydrochloride (8.5 mg) and 0.045 mmol of N-hydroxysuccinimide (5.1 mg) at room temperature. The mixture was stirred at room temperature for 5 hours to obtain a hapten activation solution.
(2) 38 mg of bovine serum albumin was dissolved in 4 mL of PBS (pH=7.4, 0.01 mol/L) to obtain a bovine serum albumin solution. Under stirring, the hapten activation solution was added to the bovine serum albumin solution. The mixture was stirred overnight at room temperature to obtain a xylazine antigen.
(3) Unreacted xylazine hapten was removed from the xylazine antigen by sufficiently dialyzing the xylazine antigen with a PBS (pH=7.4, 0.01 mol/L) solution. Then centrifugation was performed at 6000 rpm-7000 rpm for 5-6 minutes at 2° C.-8° C., the supernatant was collected to obtain a xylazine-bovine serum albumin antigen (XYL-BSA-1) of the compound of Formula (I) as the coating antigen.

### 2. Preparation of a Xylazine Immunogen of the Compound of Formula (I)

(1) 0.014 mmol of xylazine hapten (5.0 mg) of the compound of Formula (I) was dissolved in 1 mL of N,N-dimethylformamide, followed by stirring and adding 0.021 mmol of 1-(3-dimethylaminopropyl)-3-ethylcarbodiimide hydrochloride (5.0 mg) and 0.022 mmol of N-hydroxysuccinimide (2.5 mg) at room temperature, and a hapten activation solution was obtained by stirring the mixture for 5 hours at room temperature.
(2) 20 mg of keyhole limpet hemocyanin was dissolved in 4 mL of PBS (pH=7.4, 0.01 mol/L) to obtain a keyhole limpet hemocyanin solution. Under stirring, the hapten activation solution was added to the keyhole limpet hemocyanin solution. The mixture was stirred overnight at room temperature to obtain a xylazine antigen.
(3) Unreacted xylazine hapten was removed from the xylazine antigen by sufficiently dialyzing the xylazine antigen with a PBS (pH=7.4, 0.01 mol/L) solution. Then centrifugation was performed at 6000-7000 rpm for 5-6 min at 2-8° C., and the supernatant was collected to obtain a xylazine-keyhole limpet hemocyanin antigen (XYL-KLH-1) of the compound of Formula (I) as the immunogen.

### 3. Preparation of a Xylazine Coating Antigen of the Compound of Formula (II)

(1) 0.026 mmol of xylazine hapten (10.0 mg) of the compound of Formula (II) was dissolved in 1 mL of N,N-dimethylformamide, followed by stirring and adding 0.045 mmol of 1-(3-dimethylaminopropyl)-3-ethylcarbodiimide hydrochloride (8.5 mg) and 0.045 mmol of N-hydroxysuccinimide (5.1 mg) at room temperature, and a hapten activation solution was obtained by stirring the mixture for 5 hours at room temperature.
(2) 35 mg of bovine serum albumin was dissolved in 4 mL of PBS (pH=7.4, 0.01 mol/L) to obtain a bovine serum albumin solution. Under stirring, the hapten activation solution was added to the bovine serum albumin solution. The mixture was stirred overnight at room temperature to obtain a xylazine antigen.
(3) Unreacted xylazine hapten was removed from the xylazine antigen by sufficiently dialyzing the xylazine antigen with a PBS (pH=7.4, 0.01 mol/L) solution. Then centrifugation was performed at 6000-7000 rpm for 5-6 min at 2-8° C., and the supernatant was collected to obtain a xylazine-bovine serum albumin antigen (XYL-BSA-2) of the compound of Formula (II) as the coating antigen.

### 4. Preparation of a Xylazine Immunogen of the Compound of Formula (II)

(1) 0.013 mmol of xylazine hapten (5.0 mg) of the compound of Formula (II) was dissolved in 1 mL of N,N-dimethylformamide, followed by stirring and adding 0.021 mmol of 1-(3-dimethylaminopropyl)-3-ethylcarbodiimide hydrochloride (4.0 mg) and 0.022 mmol of N-hydroxysuccinimide (2.5 mg) at room temperature, and a hapten activation solution was obtained by stirring the mixture for 5 hours at room temperature.
(2) 20 mg of keyhole limpet hemocyanin was dissolved in 4 mL of PBS (pH=7.4, 0.01 mol/L) to obtain a keyhole limpet hemocyanin solution. Under stirring, the hapten activation solution was added to the keyhole limpet hemocyanin solution. The mixture was stirred overnight at room temperature to obtain a xylazine antigen.
(3) Unreacted xylazine hapten was removed from the xylazine antigen by sufficiently dialyzing the xylazine antigen with a PBS (pH=7.4, 0.01 mol/L) solution. Then centrifugation was performed at 6000-7000 rpm for 5-6 min at 2-8° C., and the supernatant was collected to obtain a xylazine-keyhole limpet hemocyanin antigen (XYL-KLH-2) of the compound of Formula (II) as the immunogen.

### Example 3: Preparation of Xylazine Antiserum

Three female Bab/c mice aged 6-8 weeks were immunized with the XYL-KLH-1 solution prepared in Example 2, and another three female Bab/c mice aged 6-8 weeks were immunized with the XYL-KLH-2 solution in Example 2. The XYL-KLH-1 solution and the XYL-KLH-2 solution were each diluted with physiological saline to a concentration of 1 mg/mL and emulsified with an equal volume of Freund's adjuvant. A first immunization was performed with Freund's complete adjuvant, with multiple subcutaneous injections at the neck and back, with a dose of 100 μg/mouse. Two weeks later, booster immunizations were performed, with one booster every 2 weeks for a total of three times. The adjuvant for the booster immunization was changed to Freund's incomplete adjuvant, with a dose of 50 μg/mouse, and the injection sites remained unchanged. One week after the final immunization, blood was collected by tail vein bleeding. After blood collection, the samples were centrifuged at 3000 rpm for 10 minutes, and the supernatant was collected as the xylazine antiserum.

### Example 4: Detection of Xylazine Antiserum

### 1. Detection of Antiserum Titer by Indirect ELISA

The coating antigen was diluted to a concentration of 2 μg/mL in PBS (8.5 g/L NaCl, 1.4 g/L Na2H PO4, 0.2 g/L NaH2PO4, pH=7.4), added to a 96-well plate, 100 μL/well, and incubated at 37° C. for 2 hours or overnight at 4° C. After washing the plate four times with Phosphate Buffered Saline with Tween 20 (PBST) (8.5 g/L NaCl, 1.4 g/L Na2HPO4, 0.2 g/L NaH2PO4, 0.5% (v/v) Tween-20, pH=7.4), the plate was tapped dry. A 1% BSA solution in PBS was added to block the plate, 200 μL/well, incubated at 37° C. for 1 hour or overnight at 4° C. The plate was washed three times with PBST and tapped dry. The xylazine antisera prepared in Example 3 was serially diluted in PBS and added to the corresponding wells, 100 μL/well. The blank control was PBS solution, and the negative control was pre-immunization mouse serum. After incubating at 37° C. for 1 hour for coating, the plate was washed. HRP-labeled goat anti-mouse IgG was diluted 1:10,000 and added to the wells, 100 μL/well. After incubating at 37° C. for 0.5 hours for coating, the plate was washed. 3,3′,5,5′-Tetramethylbenzidine (TMB) chromogenic substrate (100 μL/well) was added to each well, and incubated at 37° C. for 15 minutes. The reaction was terminated by adding 100 μL of 1M HCl to each well. OD450 nm values were measured using an enzyme-linked immunosorbent assay (ELISA) reader, and the results were observed.

### 2. Detection of Antibody Specificity

1) Coating: the coating antigens, XYL-BSA-1 and XYL-BSA-2, were diluted to 2 μg/ml with a coating buffer respectively, and 100 μL of each solution was added to each well of a 96-well plate. The plate was incubated at 37° C. for 2 hours.
2) Washing and blocking: the washing and blocking was performed in the same manner as the indirect ELISA approach described above.
(3) Preparation of a xylazine standard solution: xylazine standard was dissolved in DMF to prepare a stock solution at 5 mg/mL. Before adding samples, the stock solution was further diluted with 0.01 mol/L PBS (pH=7.4) to a required concentration.
(4) Sample addition: 50 μL of each diluted concentration of xylazine standard was added to each well, followed by the addition of 50 μL of an appropriately diluted antiserum (optimal dilution) to each well. The plate was incubated at 37° C. for 1 hour. After sufficient washing, 100 μL of HRP-labeled goat anti-mouse IgG, diluted at 1:10,000, was added to each well. The plate was incubated at 37° C. for 1 hour.
5) Color development reaction.
6) Termination and measurement: 100 μL of stop solution was added to each well to terminate the reaction. The OD450 value of each well was measured using an ELISA reader.

### 3. Experimental Results

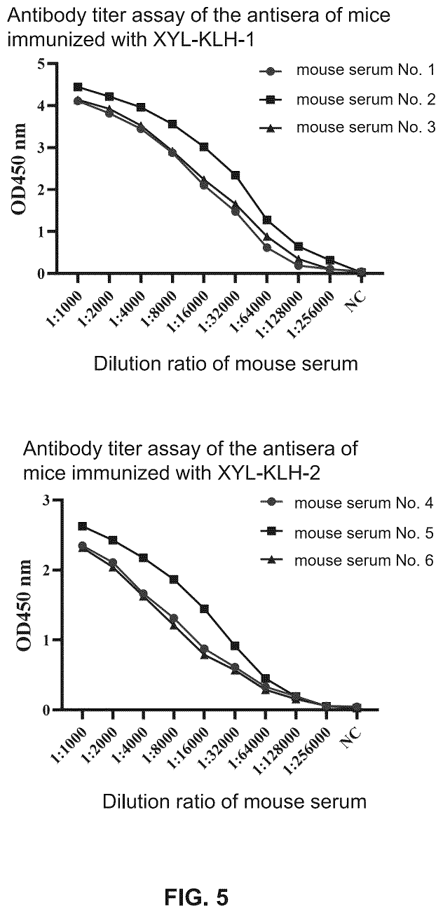

Serum titer detection results after immunizing the mice are shown in FIG. 5. The results indicate that the antisera (designated as mouse serum No. 1, mouse serum No. 2, and mouse serum No. 3) from the three mice immunized with XYL-KLH-1 exhibited excellent antibody titer. The antisera (designated as mouse serum No. 4, mouse serum No. 5, and mouse serum No. 6) from the three mice immunized with XYL-KLH-2 also showed excellent antibody titer.

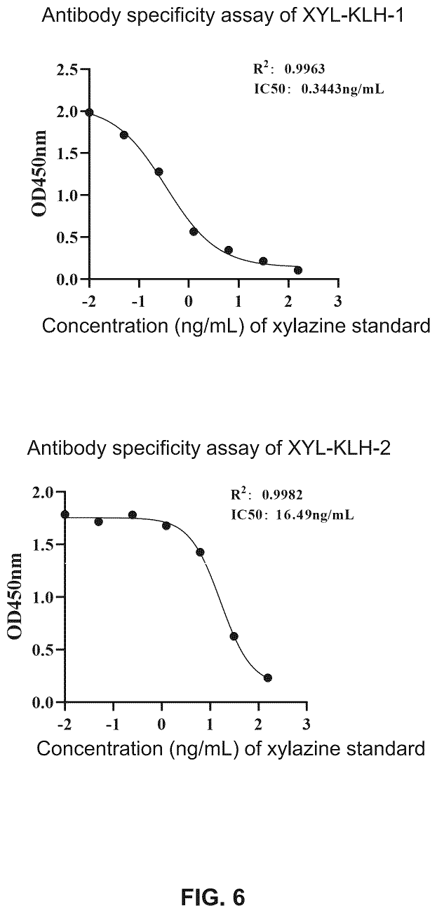

Results of the antibody specificity detection are shown in FIG. 6. The results indicate that the antibody obtained from the mice immunized with XYL-KLH-1 had a half maximal inhibitory concentration (IC50) value of 0.3443 ng/mL, demonstrating good specificity. The antibody obtained from the mice immunized with XYL-KLH-2 had an IC50 value of 16.49 ng/mL, also demonstrating good specificity.

The above descriptions of the embodiments are provided for understanding the methods of the present disclosure and the core ideas thereof, and do not constitute a limitation to the scope of the present disclosure. It should be noted that, for those skilled in the art, without departing from the principles of the present disclosure, various improvements and modifications may be made to the present disclosure. However, these improvements and modifications also fall within the scope of protection of the claims of the present disclosure.

What is claimed is:

1. A compound of Formula (I) or a compound of Formula (II) based on xylazine: 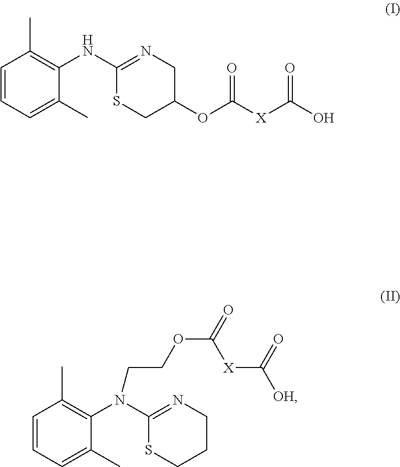
wherein X is an alkyl group having 0 to 10 carbon atoms.

2. A method for preparing a xylazine hapten of the compound of Formula (I) of claim 1, or a salt thereof, or a solvate thereof, the method comprising:
adding o-tolyl isothiocyanate to anhydrous ethanol, adding 1-amino-3-chloro-2-propanol and ethyl acetate, and extracting an intermediate product, hydroxy xylazine; and
mixing the hydroxy xylazine with a cyclic anhydride compound, followed by adding toluene, to obtain the xylazine hapten of the compound of Formula (I), or the salt thereof, or the solvate thereof.

3. The method of claim 2, wherein heating under reflux is performed after adding the 1-amino-3-chloro-2-propanol.

4. The method of claim 2, wherein after adding the toluene, heating under reflux is performed followed by concentration.

5. The method of claim 2, wherein a molar ratio of the o-tolyl isothiocyanate to the 1-amino-3-chloro-2-propanol is 1:1; the cyclic anhydride compound is a glutaric anhydride, and a mass ratio of the hydroxy xylazine to the glutaric anhydride is 1:1.

6. A method for preparing a xylazine hapten of the compound of Formula (II) of claim 1, or a salt thereof, or a solvate thereof, the method comprising:
adding xylazine and 2-iodoethanol to a N,N-Dimethylformamide (DMF) solution of K₂CO₃ to obtain an intermediate compound of Formula (III); and 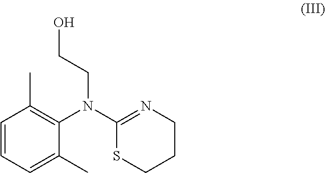 [SMILES: Cc1cccc(C)c1N(CCO)C1=NCCCS1]
mixing the compound of Formula (III) with a cyclic anhydride compound, followed by adding toluene to obtain the xylazine hapten of the compound of Formula (II), or the salt thereof, or the solvate thereof.

7. A xylazine antigen, obtained by conjugating the compound of Formula (I) or the compound of Formula (II) of claim 1 with a carrier protein.

8. The xylazine antigen of claim 7, wherein the carrier protein includes at least one selected from bovine serum albumin, ovalbumin, hemocyanin, thyroglobulin, and human serum albumin.

9. A method of preparing the xylazine antigen of claim 7, comprising:
adding a xylazine hapten to N,N-dimethylformamide, and adding 1-(3-dimethylaminopropyl)-3-ethylcarbodiimide hydrochloride and N-hydroxysuccinimide under stirring to obtain a hapten activation solution;
dissolving a carrier protein in a phosphate-buffered saline (PBS) solution to obtain a carrier protein solution, and adding the hapten activation solution to the carrier protein solution under stirring to obtain the xylazine antigen; and
removing unreacted xylazine hapten from the xylazine antigen by dialyzing the xylazine antigen with a PBS solution, centrifuging, and collecting a supernatant to obtain a xylazine-carrier protein antigen.

10. A specific antibody prepared using the xylazine antigen of claim 7, the specific antibody comprising at least one selected from a monoclonal antibody, a polyclonal antibody, a humanized antibody, a chimeric antibody, a recombinant antibody, a polyspecific antibody, a bispecific antibody, and an antibody fragment.

11. A method for diagnosing whether xylazine is present in the body of a patient, comprising: contacting the specific antibody of claim 10 with a sample of the patient, and determining whether xylazine is present in the body of the patient by determining whether a xylazine-antibody complex is generated.

12. A method for treating a patient containing xylazine, comprising: administering to the patient a therapeutically effective amount of the specific antibody of claim 10.

13. A method for preparing an anti-xylazine specific antibody, comprising: stimulating a subject using the compound of Formula (I) or the compound of Formula (II) of claim 1, and collecting the anti-xylazine specific antibody from the subject.

14. A method for preparing an anti-xylazine specific antibody, comprising: stimulating a subject using the xylazine antigen of claim 7, and collecting the anti-xylazine specific antibody from the subject.

15. A method for detecting an anti-xylazine specific antibody, comprising: contacting the anti-xylazine specific antibody with the compound of Formula (I) or the compound of Formula (II) of claim 1, and detecting an amount of the anti-xylazine specific antibody based on a concentration of a generated hapten-antibody complex.

16. A method for detecting an anti-xylazine specific antibody, comprising: contacting the anti-xylazine specific antibody with the xylazine antigen of claim 7, and detecting an amount of the anti-xylazine specific antibody based on a concentration of a generated antigen-antibody complex.

17. A composition for preparing a product for detecting xylazine, the composition comprising the compound of Formula (I) or the compound of Formula (II) of claim 1.

18. A composition for preparing a product for detecting xylazine, the composition comprising the xylazine antigen of claim 7.

19. A composition for preparing a product for detecting xylazine, the composition comprising the specific antibody of claim 10.

20. A method for detecting xylazine, comprising: contacting a test subject with the specific antibody of claim 10, and determining whether the test subject contains the xylazine by determining whether a xylazine-antibody complex is generated.
